(19) United States
(12) Patent Application Publication
(10) Pub. No.: US 2004/0034298 A1
(43) Pub. Date: Feb. 19, 2004

(54) Radiographic sizing tool

(75) Inventors: Brooks A. Johnson, Minnetonka, MN (US); Jeffrey A. Sarge, Fremont, CA (US); Jeremy J. Gillis, Maple Grove, MN (US); Dennis B. Werner, Big Lake, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: SCIMED LIFE SYSTEMS, INC.

(21) Appl. No.: 10/217,970

(22) Filed: Aug. 13, 2002

(51) Int. Cl. A61B 5/05

(52) U.S. Cl. 600/407

(57) ABSTRACT

A radiographic sizing tool comprising a radiotranslucent carrier with a plurality of spaced apart radiopaque objects disposed therein. The radiopaque objects have the same shape and dimension throughout at least two different planes of view, and preferably all planes of view, to reduce parallax during radiography. For example, the radiopaque objects may comprise spheres having different diameters, preferably in uniform increments and arranged in order of (increasing or decreasing) diameter. The radiographic sizing tool is particularly beneficial in angiography procedures for sizing coronary vessels and stents.

### FIELD OF THE INVENTION

The disclosure herein generally relates to devices used in radiography. In particular, the disclosure herein relates to sizing tools used in radiographic procedures such as angiography.

### BACKGROUND OF THE INVENTION

Angiography is an x-ray radiographic visualization technique used to produce images of the heart and associated anatomy to facilitate diagnostic and/or therapeutic procedures such as angioplasty and stenting. In such procedures, it is important to accurately determine the size of the vascular lumen such that the correct balloon size and/or stent size may be selected. However, current methods of determining the correct size of the vascular lumen are subject to substantial error, and/or are cost prohibitive to implement.

For example, interpolating (i.e., “eyeballing”) the size of the vascular lumen by comparison to the size of the guide catheter may be subject to substantial human error. Other interpolation techniques which utilize radiopaque objects such as rulers, coins and washers of known size for comparison to the vascular lumen are also subject to human error, and further introduce the potential for parallax error since the radiopaque objects are substantially planar. Quantitative Coronary Angiography (QCA) may reduce some of the human error, but QCA is cost prohibitive to implement because it requires additional capital equipment, additional staff to operate, and additional procedure time. Thus, there is an ongoing need for less expensive and more accurate techniques for sizing vascular lumens.

### SUMMARY OF THE INVENTION

To address this ongoing need, the present invention provides a radiographic sizing tool comprising, in one example, a plurality of spaced apart radiopaque objects disposed in a radiotranslucent carrier. The radiopaque objects have the same shape and dimension (e.g., spheres) throughout at least two different planes of view, and preferably all planes of view, to reduce parallax. The radiopaque spheres may have different diameters, preferably in uniform increments and arranged in order of increasing or decreasing diameter, to assist in sizing anatomical features such as vascular lumens.

### BRIEF DESCRIPTION OF THE DRAWINGS

FIG. 1 is a top view of a radiographic sizing tool;

FIG. 2 is an end view of the radiographic sizing tool shown in FIG. 1;

FIG. 3 is a top view of another radiographic sizing tool;

FIG. 4 is an end view of the radiographic sizing tool shown in FIG. 3;

FIG. 5 is a side view of the radiographic sizing tool shown in FIG. 3; and

FIG. 6 is a top view of yet another radiographic sizing tool.

### DETAILED DESCRIPTION OF THE INVENTION

The following description should be read with reference to the drawings wherein like reference numerals indicate like elements throughout the several views. The detailed description and drawings illustrate embodiments by way of example, not limitation.

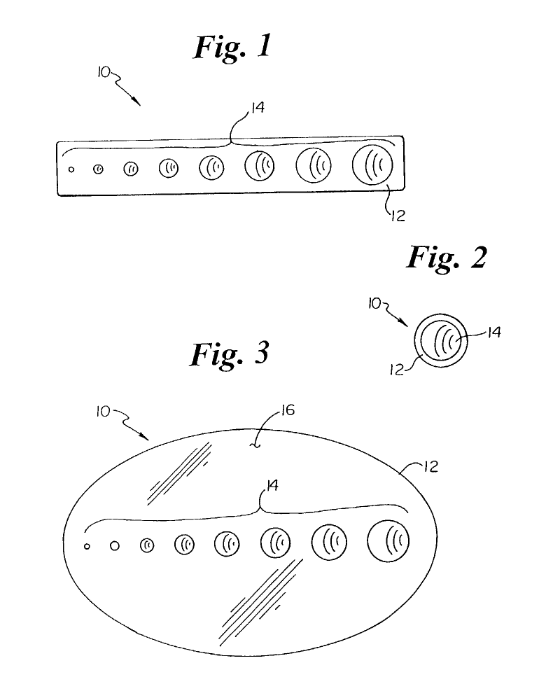

Refer now to FIG. 1 which illustrates a radiographic sizing tool 10 in accordance with one embodiment of the present invention. Radiographic sizing tool 10 includes a carrier 12 and a plurality of radiopaque objects 14 disposed therein. Carrier 12 may comprise any suitable structure for holding the radiopaque objects 14.

By way of example, not limitation, the carrier 12 may comprise a moldable material which encapsulates the radiopaque objects 14. The radiopaque objects 14 may be placed into a mold and a moldable material (e.g., thermoplastic polymer, curable resin, curable gel, etc.) may be injected into the mold and around the radiopaque objects 14 to form a carrier 12 that encapsulates and retains the radiopaque objects 14 therein. Alternatively, the radiopaque objects 14 may be secured to the carrier by an adhesive or an adhesive tape.

As a further alternative, the radiopaque objects 14 may be placed in a moldable material that thermally forms around the radiopaque objects 14. For example, the radiopaque objects 14 may be placed in a tube comprising a heat shrinkable material and subsequently exposed to heat such that the material shrinks onto the radiopaque objects 14 to form a carrier 12. The radiopaque objects 14 may alternatively be placed in a tube comprising a thermoplastic material and subsequently exposed to heat and a vacuum such that the material shrinks onto the radiopaque objects 14 to form a carrier 12.

The material forming the carrier 12 may comprise a radiotranslucent material such that the material does not compromise visualization of the radiopaque objects 14 during radiography, and to provide contrast to the radiopaque objects 14. Most polymeric materials, absent radiopaque loading, are sufficiently radiotranslucent to provide this effect. The material forming the carrier 12 may also comprise a material capable of withstanding sterilization processes, such as conventional medical grade plastics. To permit visual inspection of the radiopaque objects 14 in the carrier, the carrier 12 may be formed of a transparent or semi-transparent material.

The carrier 12 may be sized to accommodate a plurality of radiopaque objects 14 that are generally spherical. Accordingly, the carrier 12 may be elongate as shown in FIG. 1, although any shape that is sized to accommodate the radiopaque objects 14 and is easy to handle in-vitro may be utilized. For example, the carrier 12 may be cylindrical as shown in FIGS. 1 and 2 with a length of approximately 5 cm and a diameter of approximately 6-8 mm. Alternatively, the carrier 12 may be ellipsoidal in shape as shown in FIG. 3, with a major diameter of approximately 5 cm and a thickness of approximately 6-8 mm. As a further alternative, the carrier 12 may be circular in shape as shown in FIG. 6, with a diameter of approximately 5 cm and a thickness of approximately 6-8 mm.

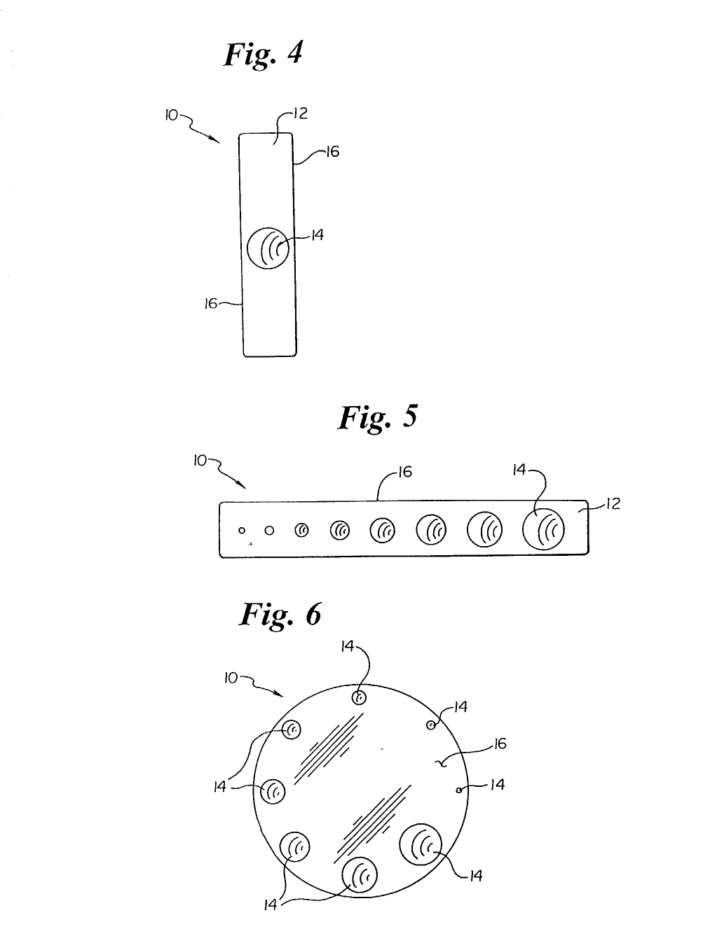

The carrier 12 may have a circular profile as best seen in FIG. 2, or a flat profile having a major flat side or surface 16 as shown in FIGS. 4, 5 and 6. The flat side(s) 16 of the carrier 12 minimizes the risk of movement (due to rolling) of the tool 10 when placed on the patient's body. To further reduce the risk of movement when placed on the patient's body, the carrier 12 may be formed of a conformable material (e.g., cured and cross-linked gel) to permit the tool 10 to conform to body surface contours of the patient. In addition, an adhesive backing may be applied to the back surface 16 of the carrier 12 to provide a similar effect.

The radiopaque objects 14 may have the same dimension throughout at least two different planes of view to reduce parallax error during radiography. This is particularly beneficial when the radiography procedure involves x-ray images taken in two or more different planes of view, which may introduce parallax error if planar radiopaque objects are used. To this end, the radiopaque objects 14 may have a spherical or semi-spherical shape, for example.

The radiopaque objects 14 may have differing sizes (e.g., diameters) selected as a function of the anatomy being sized, and may be uniformly spaced apart in the carrier 12. The radiopaque objects 14 may be arranged in order of increasing or decreasing diameter in the carrier 12, and the diameters may increase or decrease in uniform increments. The diameters may range from 1 mm to 12 mm for typical peripheral vascular applications, and may have diameters in the range of 1 mm to 6 mm for typical coronary and neuro vascular applications. The uniform increments may be in whole or fractional millimeter units (e.g., half millimeter units: 1 mm, 1.5 mm, 2.0 mm, 2.5 mm, 3.0 mm, 3.5 mm, 4.0 mm, etc.), or whole French units, for example, which are conventional dimensions used in sizing vascular lumens, balloons, stents and other medical devices. Given the desirability for precise measurement, the radiopaque objects 14 may have a size tolerance of +/−0.0005 mm to +/−0.005 mm, and/or no more than +/−1%.

The radiopaque objects 14 may comprise a material that provides adequate opacity for x-ray visualization. Most dense metals and metal alloys such as stainless steel, platinum, platinum iridium, gold, brass, etc., may be used to provide this effect. Those skilled in the art will recognize that other suitable materials may be used such as polymeric materials loaded with radiopaque filler.

The radiographic sizing tool 10 may be utilized in an otherwise conventional radiography procedure to obtain images of a patient's anatomy (e.g., coronary, neuro, or peripheral vasculature) and to determine the size of a particular feature of the anatomy. In use, the radiographic sizing tool 10 is placed on the patient's body proximate the anatomy of interest and within the x-ray field. For example, the tool 10 may be placed in the supine position on the patient's chest for angiography of the coronary vasculature, on the patient's head or neck for radiography of the neuro vasculature, or on the patient's extremities (arms or legs) for radiography of the peripheral vasculature.

Once the radiographic sizing tool 10 is in the desired position on the patient's body, an x-ray image is taken. Because the radiographic sizing tool 10 is in the x-ray field, the image will contain both the anatomy of interest and the radiopaque objects 14. Typically, two or more x-ray images are taken in at least two different planes of view, which would introduce parallax error absent the unique shape (e.g., spherical or semispherical) of the radiopaque objects 14.

After an image is produced, the radiopaque objects 14 may be compared to an anatomical feature of interest. During this comparison, the radiopaque object 14 that most closely matches the size of the anatomical feature is identified. Because the size of the radiopaque objects 14 are known, the size of the anatomical feature may be determined by correlation.

As mentioned previously, the anatomical feature may comprise a vascular lumen, such as a vascular lumen of a coronary, neuro or peripheral vessel. Alternatively, the same technique may be used to determine the size of a device implanted or otherwise disposed in the patient. For example, the same technique may be used to determine the size of a stent disposed in a vascular lumen, or the patency of the lumen extending through the stent.

Those skilled in the art will recognize that the present invention may be manifested in a variety of forms other than the specific embodiments described and contemplated herein. Accordingly, departures in form and detail may be made without departing from the scope and spirit of the present invention as described in the appended claims.

What is claimed is:

1. A method of performing a radiography procedure to obtain images of a patient's anatomy, the method comprising:
providing a radiographic sizing tool comprising a radiotranslucent carrier with a plurality of spaced apart radiopaque objects disposed therein, wherein the objects have the same dimension throughout at least two different planes of view to reduce parallax during radiography;
placing the radiographic sizing tool on the patient's body proximate the anatomy of interest; and
producing an x-ray image containing the anatomy of interest and the radiopaque objects.

2. A method of performing a radiography procedure as in claim 1, wherein at least two x-ray images are produced in at least two different planes of view.

3. A method of performing a radiography procedure as in claim 1, further comprising:
comparing the radiopaque objects to a feature of the anatomy; and
identifying the radiopaque object that most closely matches the size of the feature of the anatomy.

4. A method of performing a radiography procedure as in claim 3, wherein the feature comprises a vascular lumen.

5. A method of performing a radiography procedure as in claim 3, wherein the feature comprises a vascular lumen of a coronary vessel.

6. A method of performing a radiography procedure as in claim 3, wherein the feature comprises a vascular lumen of a neuro vessel.

7. A method of performing a radiography procedure as in claim 3, wherein the feature comprises a vascular lumen of a peripheral vessel.

8. A method of performing a radiography procedure as in claim 1, further comprising:
comparing the radiopaque objects to a device disposed in the anatomy; and
identifying the radiopaque object that most closely matches the size of the device disposed in the anatomy.

9. A method of performing a radiography procedure as in claim 8, wherein the device comprises a stent disposed in a vascular lumen.

10. A method of performing a radiography procedure as in claim 1, wherein the step of placing the radiographic sizing tool on the patient's body comprises placing the radiographic sizing tool on the patient's chest.

11. A method of performing a radiography procedure as in claim 1, wherein the step of placing the radiographic sizing tool on the patient's body comprises placing the radiographic sizing tool on the patient's head or neck.

12. A method of performing a radiography procedure as in claim 1, wherein the step of placing the radiographic sizing tool on the patient's body comprises placing the radiographic sizing tool on one of the patient's extremities.

13. A method of performing a radiography procedure as in claim 1, wherein the radiotranslucent carrier has a flat major surface, and wherein the step of placing the radiographic sizing tool on the patient's body comprises placing the flat major surface of the radiotranslucent carrier on the patient's body.

14. A method of performing a radiography procedure as in claim 13, wherein the radiotranslucent carrier is conformable, and wherein the step of placing the radiographic sizing tool on the patient's body comprises conforming the radiotranslucent carrier to the patient's body surface contour.

15. A radiographic sizing tool, comprising:
a radiotranslucent carrier; and
a plurality of spaced apart radiopaque objects disposed in the carrier, wherein the objects have the same dimension throughout all planes of view.

16. A radiographic sizing tool as in claim 15, wherein the plurality of radiopaque objects have the same dimension throughout all planes of view.

17. A radiographic sizing tool as in claim 16, wherein the plurality of radiopaque objects comprises spheres.

18. A radiographic sizing tool as in claim 17, wherein the plurality of radiopaque spheres has different diameters.

19. A radiographic sizing tool as in claim 18, wherein the plurality of radiopaque spheres are arranged in order of increasing or decreasing diameter.

20. A radiographic sizing tool as in claim 19, wherein the different diameters are in uniform increments.

21. A radiographic sizing tool as in claim 20, wherein the diameters are in the range of 1 mm to 6 mm.

22. A radiographic sizing tool as in claim 20, wherein the uniform increments are in whole millimeter units.

23. A radiographic sizing tool as in claim 20, wherein the uniform increments are in half-millimeter units.

24. A radiographic sizing tool as in claim 20, wherein the uniform increments are in whole French units.

25. A radiographic sizing tool as in claim 17, wherein the plurality of radiopaque spheres comprises a metal.

26. A radiographic sizing tool as in claim 25, wherein the carrier comprises a polymer.

27. A radiographic sizing tool as in claim 26, wherein the carrier is sized for in-vitro handling.

28. A radiographic sizing tool as in claim 27, wherein the polymer is relatively transparent to visible light such that the plurality of radiopaque spheres are visible therethrough.

29. A radiographic sizing tool as in claim 27, wherein the carrier has a flat major side.

30. A radiographic sizing tool as in claim 29, wherein the carrier is conformable to body contours.

31. A radiographic sizing tool, comprising:
a radiotranslucent carrier; and
a plurality of spaced apart radiopaque objects disposed therein, wherein the objects have means for reducing parallax during radiography.

32. A radiographic sizing tool as in claim 31, wherein the parallax reducing means comprises a shape of the radiopaque objects.

33. A radiographic sizing tool as in claim 32, wherein the shape has the same dimension throughout at least two different planes of view.

34. A radiographic sizing tool as in claim 33, wherein the shape is spherical.
